(19) United States
(12) Patent Application Publication
(10) Pub. No.: US 2007/0134694 A1
(43) Pub. Date: Jun. 14, 2007

(54) Method for the measurement of dengue virus binding inhibition

(76) Inventors: Timothy Burgess, Silver Spring, MD (US); Jeffrey A. Tjaden, Eagle River, AK (US); Kevin Porter, Boyds, MD (US); Nicole Couillard Martin, Olney, MD (US); Mary Marovich, Bethesda, MD (US)

Correspondence Address: NAVAL MEDICAL RESEARCH CENTER;ATTN: (CODE 00L), 503 ROBERT GRANT AVENUE, SILVER SPRING, MD, 20910-7500, US

(21) Appl. No.: 11/586,894

(22) Filed: Oct. 26, 2006

Related U.S. Application Data
(60) Provisional application No. 60/731,990, filed on Oct. 27, 2005.

(51) Int. Cl. C12Q 1/70; C12Q 1/68

(52) U.S. Cl. 435/6; 435/5

(57) ABSTRACT

The invention concerns a method of detecting dengue infection by detecting anti-dengue neutralizing substances including anti-dengue antibody by measuring inhibition of dengue binding to dentritic cell-specific intracellular adhesion molecule (ICAM) 3-grabbing nonintegrin (DC-SIGN) or liver/lymph node-specific intracellular adhesion molecule (ICAM) 3-grabbing nonintegrin (L-SIGN). DC-SIGN or L-SIGN can be used expressed in transfected cell lines that normally are not infected by dengue virus. The invention also concerns a method of detecting anti-dengue drugs by their ability to inhibit binding of dengue virus to DC-SIGN or L-SIGN.

### CROSS-REFERENCE TO RELATED APPLICATIONS

This application claims priority to U.S. Provisional application 60/731,990 filed Oct. 27, 2005.

### FIELD OF INVENTION

The inventive subject matter relates to a method of detecting dengue virus binding inhibitory substances, including anti-dengue virus antibody, in human serum by immunocytometry.

### BACKGROUND OF INVENTION

1. Field of Invention

The inventive subject matter relates to a method of detecting dengue virus infection by detecting inhibiting substances, such as anti-dengue antibody in human serum by immunocytometry. The inventive subject matter also relates to a method of detecting anti-dengue drugs.

2. Background Art

Dengue virus, the causative agent of dengue fever and dengue hemorrhagic fever (DHF), is a virus of the genus Flavivirus, a single-stranded enveloped RNA virus. Dengue fever is the most common human arbovirus infection worldwide and serious public health concern accounting for estimates of 100 million infections annually (WHO 1986; Monath and Heinz 1996; Thomas, et al 2003). Dengue and DHF is found in most tropical areas including Africa, Asia, the Pacific, Australia and the Americas.

Although the virus is capable of growing in a variety of species of mosquitoes, including Aedes albopictus, Aedes polynesiensis and Aedes scutellaris, Aedes aegypti is the most efficient of the mosquito vectors because of its domestic habitat (Gubler 1988). Four antigenically distinct serotypes of dengue virus have been identified with all causing human diseases (Gubler, et al 1979; Henchal and Putnak 1990). Each of the four serotypes, although distinct, is similar enough to the others to elicit only partial cross-protection following infection (WHO 1986). Following infection, viremia is typically detected early at the onset of symptoms (Halstead 1997). Although many infections are mild, some infections result in dengue hemorrhagic fever and dengue shock syndrome, which are potentially fatal. This usually occurs in a small number of people during a second infection with a dengue virus that is different from the virus causing the first infection (Halstead 1997).

Dengue virus infection occurs following the bite of dengue virus-infected Aedes mosquitoes, which in-turn were infected by prior feeding on infected humans. Symptoms of dengue infection, including high fever, severe headache, retro-orbital pain, development of a rash, nausea, joint and muscle pain, usually start within five to six days following the bite of an infected mosquito. The fatality rate is 6 to 30% with most deaths occurring in infants. The management of DHF is symptomatic and supportive and aimed at replacement of plasma loss (Nimmannitya 1996).

It is not possible to make an accurate diagnosis of mild or classical dengue fever based on clinical features of dengue fever since many symptoms of dengue fever resemble those of other diseases, such as chikungunya infection (Nimmannitya 1996), measls, influenza and rickettsial infections. Differential diagnosis must include malaria and other viral, bacterial and rickettsial diseases. Diagnostic methods for infection are typically based on detection of virus, viral antigens genomic sequencing and dengue-specific antibodies (Shu and Huang 2004). Dengue hemorrhagic fever (DHF) can, however, be more accurately diagnosed from clinical symptoms, including high continuous fever for 2 to 7 days, hepatomegaly and shock, thromocytopenia. In diagnosis of dengue fever or DHF, it is usually necessary to base the diagnosis on results using a combination of methods. Even confirmation of dengue hemorrhagic fever requires serological analysis for assessment of seroconversion or virus isolation.

Antibodies to dengue virus antigens increase rapidly in patients with secondary dengue infection. A diagnostic (typically four-fold) increase in dengue antibody can often be observed during febrile illness. However, in primary dengue infection, specimens are usually acquired as late as two to three weeks after onset of disease. Confirmatin of seroconversion by antibody detection requires two samples, one at the beginning of the manifestation of clinical symptoms and the other 10 to 28 days later. The method of detecting antibody is usually via haemaglutination reaction (IHA) or by ELISA. For example, U.S. Pat. No. 5,824,506 to Chan, et al (1998) discloses a method for detecting antibodies to dengue in serum from infected patients using peptide antigens derived from dengue virus type-2 NS 1. However, this method may suffer from disadvantages including the fact that the selected peptides may not offer sensitive recognition of antibodies, especially early after infection.

Laboratory diagnostic methods require a rapid turn-around from sample collection to result and the ability to screen large numbers of samples, accurately. The current “gold standard” method for detecting acute dengue infection involves virus isolation and characterization using virus culture and growing the virus in a susceptible cell line such Vero cells, an the African Green monkey kidney cell line or the mosquito line C6/36, derived from the mosquito Aedes albopictus. Although the assay method is considered reliable, it suffers from serious disadvantages.

Most importantly, the plaque reduction neutralization test (PRNT) assay suffers from a relatively low sensitivity and significant time, five to ten days, before results are available. Use of flow cytometry, however, has been shown to be capable of identifying intracellulary antigen in a specific and sensitive fashion (Darden, et al 2000; Mascola, et al 2002). Furthermore, results using the PRNT may not be relevant to human dengue infection because the test uses highly passaged, laboratory-adapted viral stocks and dengue susceptible substrates such as African green monkey kidney cells (Vero) or baby hamster kidney (BHK) cells. It is possible, as shown for other viruses, including dengue, that serial passages result in mutations, altering cell receptor binding such as demonstrated in measles virus by Hsu, et al (J. Virol 1998). Also, measuring the reduction of dengue infection in animal renal epithelial cell lines may not be applicable to protection or susceptibility in humans. This is especially true since the target cells typically used are not those cell types thought to be the primary infection targets in humans. Therefore, the PRNT assay may not accurately reflect nor predict in vivo biological activity of antibodies in humans.

In measles, it has recently been discovered that the Vero-adapted measles receptor, CD46, appears to be irrelevant for wild-type measles virus infection. The Edmonston measles virus, a laboratory strain of measles, readily acquires a single amino acid change in the hemagglutinin protein upon serial passaging in Vero cells that confers its ability to bind CD46 (Hsu, et al, 1998). Wild-type isolates of measles virus from clinical isolates, however, have little affinity for CD46. Instead, CD150 (signaling lymphocytic activation molecule, SLAM) was discovered to mediate wild-type measles infection (Hsu et al, 2001). In addition, the different receptor usage affected neutralization, as antibodies directed against CD46 had no effect on wild-type infections of marmoset B cells and only partially inhibited the replication of the Edmonston laboratory strain in the same cells (Hsu et al, 1998). Polack, et al (2003) studied immune responses in macaques receiving either a live measles vaccine or a formalin-inactivated, alum-precipitated vaccine (FIMV). Post-vaccination, pre-challenge serum neutralizing titers measured in a conventional Vero PRNT were indistinguishable between the two groups. However, only the macaques that received the live measles vaccine were protected from challenge. Although the conventional PRNT could not distinguish the two groups, a urea elution ELISA measuring the avidity of anti-measles antibodies did distinguish between them (avidity was significantly lower after FIMV than after live vaccine). Most notably, measles neutralization was lower in serum from FIMV than live virus vaccinated animals when measured in an alternative assay using a marmoset B cell line infected via SLAM, the receptor felt by some to be more important for wild-type measles isolates. This result correlated well with the avidity results from the ELISA and with protection in the measles system.

Because of the interest in designing effective therapies and diagnostic methods against dengue virus invention, considerable effort has been direct toward the mechanisms of dengue virus infection. It was reported that dendritic cells utilize a dendritic cell-specific intracellular adhesion molecule (ICAM) 3-grabbing nonintegrin (DC-SIGN), a C-lectin, to promote efficient infection in trans of cells that express CD4 and chemokine receptors (Geijtenbeek, et al 2004). Flow cytometric analysis of the hematopoetic cells using anti-DC-SIGN antibodies demonstrated that DC-SIGN is preferentially expressed on in vitro cultured DC but not on other leukocytes, such as monocytes and peripheral blood lymphocytes (Geijtenbeek, et al., 2000). Amino acid analysis of the 44 kDa protein has shown it to be identical in amino acid sequence to gp120-binding C-type lectin isolated from a placental cDNA library (Curtis, et al. 1992).

It was demonstrated that subsets of dentritic cells susceptible to infection with dengue virus in culture express DC-SIGN (Marovich, et al 2001; Libraty, et al 2001). DC-SIGN also permits infection of dentrictic cells by other virus including cytomegalovirus (CMV), Ebola virus, hepatitis C virus, HIV, Marburg virus and SARS coronavirus (Alvarez, et al, 2002; Baribaud, et al, 2002; Halary, et al, 2002; Colmenares, et al, 2002; Maeda, et al, 2003; Tailleux, et al, 2003). It was also recently reported that dengue virus infection directly correlates with expression of DC-SIGN or its homologue liver/lymph node-specific specific intracellular adhesion molecule (ICAM) 3-grabbing nonintegrin (L-SIGN) (Tassaneetrithep, et al, 2003).

### SUMMARY OF INVENTION

Currently available methods for the detection and diagnosis of dengue infection early after infection are relatively insensitive and require considerable time prior to receiving results. Therefore, an object of this invention is a method of detecting dengue virus infection by detecting dengue/DC-SIGN or L-SIGN binding neutralizing antibody in patient sera.

Another object of the invention is a method of detecting dengue/DC-SIGN or L-SIGN neutralizing antibody in patient serum using DC-SIGN L-SIGN transfected cell lines.

Another object of the invention is a method of detecting dengue/DC-SIGN or L-SIGN inhibiting substances such as anti-dengue drugs.

Another object of the invention is a method of diagnosing dengue virus infection by detecting dengue/DC-SIGN or L-SIGN neutralizing antibody bound dengue virus by flow cytometery.

### BRIEF DESCRIPTION OF DRAWINGS

FIG. 1. Neutralization of infection by anti-dengue sera.

FIG. 2. Graphical comparison of effectiveness and specificity of neutralization of anti-dengue antibody compared to negative control.

### DESCRIPTION OF THE PREFERRED EMBODIMENTS

Important limitations exit for currently available methods analyzing patient serum, such as PRNT assay. Therefore, assays capable of analyzing large numbers of patient serum for diagnosis of dengue infection is needed. The current invention incorporates the ability of DC-SIGN and L-SIGN to mediate dengue infection 5 (Tassaneetrithep, et al, 2003) into a virus neutralization assay. Table 1 illustrates the ability of DC-SIGN to facilitate infection into a Raji B cell line. In Table 1, infectivity was assessed by flow cytometry as a function of varying inputs of virus and time of harvesting infected cells. P values were obtained from a Student's t test for independent samples against mock-infected cells.

| | MOI^{a} | | | | | |
|---|---|---|---|---|---|---|
| | 0.0005^{b} | | 0.0001 | | 0.00001 | |
| | | P value | | P value | | P value |
| Dengue 1 | | | | | | |
| 48 hrs | 13.3% | <0.001 | 3.3% | 0.003 | 0.2% | 0.091 |
| 72 hrs | 39.0% | <0.001 | 26.3% | <0.001 | 7.3% | <0.001 |
| 96 hrs | 42.5% | <0.001 | 30.4% | 0.005 | 14.8% | <0.001 |
| Dengue 2 | | | | | | |
| 48 hrs | 12.3% | 0.682 | 2.6% | 0.11 | 0.1% | 0.102 |
| 72 hrs | 31.2% | <0.001 | 14.3% | 0.003 | 2.1% | 0.111 |
| 96 hrs | 44.3% | <0.001 | 35.8% | <0.001 | 6.9% | <0.001 |
| Dengue 3 | | | | | | |
| 48 hrs | 1.0% | 0.2 | 0.1% | 0.149 | 0.0% | — |
| 72 hrs | 8.9% | <0.001 | 2.0% | <0.001 | 0.6% | 0.001 |
| 96 hrs | 25.3% | <0.001 | 8.3% | 0.002 | 1.5% | 0.004 |
| Dengue 4 | | | | | | |
| 48 hrs | 10.8% | <0.001 | 2.2% | 0 | 0.0% | 0.22 |
| 72 hrs | 47.1% | <0.001 | 39.1% | <0.001 | 4.3% | 0.37 |
| 96 hrs | 52.1% | <0.001 | 42.2% | <0.001 | 25.7% | <0.001 |

^{a}MOI = multiplicity of infection.
^{b}MOI of 0.001 corresponds to an input of 60 plaque forming units (PFU).

The inventive method is predicated on the reduction in infectivity of cells expressing DC-SIGN or L-SIGN. Normally dengue infection-insensitive tumor cells, such as Raji B cells can be transfected with DC-SIGN making them high susceptible to dengue infection. Raji B cells expressing DC-SIGN rapidly accumulate easily detectable amounts of dengue antigen. Therefore, discrimination between infection and non-infection is readily discemable within 24 hours (Tassaneetrithep, et al, 2003). By utilizing millions of cells with each representing the analog of one potential plaque, the sensitivity of the assay is increased dramatically.

The inventive method contemplates any method for measurement of dengue infection, including microscopy. However, a preferred embodiment is the measurement of anti-dengue antibody binding by flow cytometry. Flow cytometry is an extremely sensitive measurement technique and further improves assay reliability and amenable to high-throughput analysis. Additionally, since only small amounts of serum are required, conservation of resources is improved over currently available methods such as PRNT.

The general inventive method comprises the following steps:
a. serial dilutions of test sera and control (non-immune) sera are added to inocula of virus at a dose pre-determined to yield detectable infectivity;
b. incubate sera/virus mixture for 30 minutes;
c. add incubated sera/virus mixture to DC-SIGN transfected cells cultured into 96-well plates;
d. incubate virus/sera added transfected cells overnight;
e. measure intracellular dengue antigen 20 to 24 hours after infection;
f. from measurement of intracellular dengue antigen, determine the percent neutralization of infection of the sera dilutions based on control samples.

The percent reduction of infection at each dilution is calculated as the number of positive cells from normal sera sample minus the number of positive cells from the test sample divided by the number of positive cells from the normal sera sample multiplied by 100. The resultant is a reduction expressed as a percentage. In this manner, any non-specific neutralization of normal sera is subtracted out and baseline reduction is set at 0.

A reduction of infection at each dilution is analyzed by probit analysis/best-fit quadratic estimation and curve fitting non-linear regression analysis. Because some cells, such as Raji B cells, express FcR, it is possible that functional internalization of dengue virus complexed to non-neutralizing antibody may be measurable simultaneously with neutralization of infectivity. Therefore, the net effect is dependent solely upon the characteristics of the particular serum specimen analyzed. The estimation of net enhancement of infection verses net neutralization of infection can be determined mathematically.

Example of Neutralization using Anti-dengue and Anti-flavivirus Antibody

As a specific example, neutralization of specific anti-dengue antibody (3H5) compared to anti-flavivirus antibody (4G2) and the negative control antibody 15F3 using pRc/CMV-DC-SIGN transfected Raji B cells (Geijtenbeek, et al, 2000). Measurement of dengue infection was by flow cytometry.

The assay was conducted in 96-well plates in duplicate. Neutralizing sera and normal sera control was 4-fold serially diluted in complete growth media (GM)(RPMI 1640 supplemented with L-glutamine, fetal bovine serum (FBS) and penicillin and streptomycin. Dengue virus is added to the sera dilutions. The virus is used at a dilution to yield a multiplication of infection (MOI) that will result in an approximately 10-20% infection rate. The plates were then incubated at 37° C. under 5% CO₂ for 30 minutes. In addition to test samples, control samples were prepared containing sera plus media (in place of virus). Additionally, controls samples for back-tritration were prepared containing several dilutions starting at the 1:10 dilution viral dilution yielding an MOI giving 10-20% infection rate plus media. Subsequent to incubating sera and virus, DC-SIGN transfected Raji B cells were seeded at 1.2×10⁵ per well. The DC-SIGN transfected Raji B cells, sera and virus was incubated for 24 hours at 5% CO₂ at 37° C.

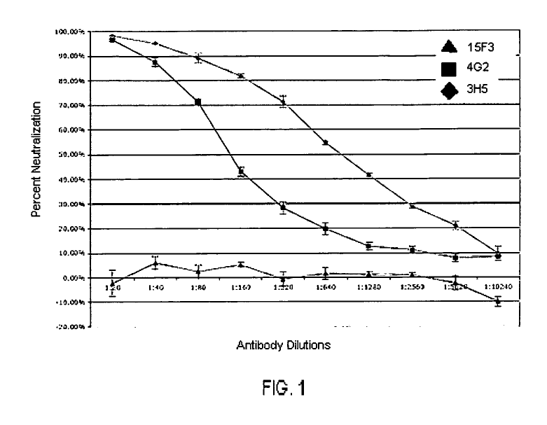

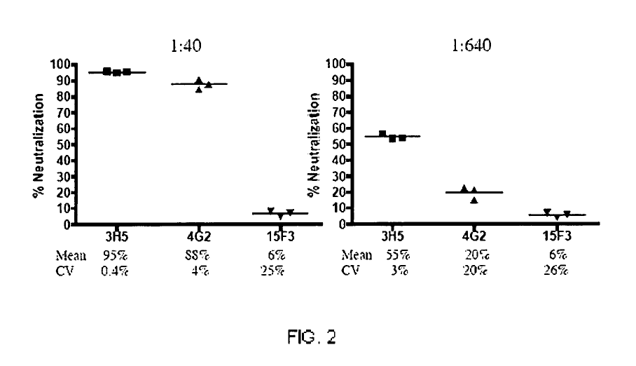

Infectivity was analyzed by measuring intracellular preM-antigen by flow cytometry. Intracellular dengue virus antigen was detected by first fixing cells with 4% paraformaldehyde, permeabilitzed with 0.5% saponin using direct staining with antibody (e.g. anti-dengue 2, 3H5 or anti-flavivirus, 4G2 or non-neutralizing antibody, 15SF3). Samples were analyzing by acquiring approximately 10,000 events per sample. The control (uninfected) samples were used as background reference and a graphical representation of the percentage of infection verses associated virus dilution was determined. A plot is then constructed whereby percent infection is a function of increasing MOI. FIG. 1 shows results of this experiment as percent neutralization verses antibody dilution. FIG. 2 illustrates the effectiveness and specificity of neutralization (e.g. compare the anti-dengue 3H5 with anti-flavivirus 4G2) of two dilutions of antibody compared to negative control.

This example illustrates the use of the current inventive method using Raji B cell tumor and flow cytometry. However, it is recognized that other the assay may be used by neutralizing the infectivity of dengue to other cell lines transfected with DC-SIGN. It is also contemplated as part of the current invention that DC-SIGN can be attached to other surfaces such as microbeads. In this case the percent neutralization is the inhibition of binding of dengue to immobilized DC-SIGN.

### REFERENCES

1. Alvarez, C. P., F. Lasala, J. Carrillo, O. Muniz, A. L. Corbi, and R. Delgado. 2002. C-type lectins DC-SIGN and L-SIGN mediate cellular entry by Ebola virus in cis and in trans. J. Viro. 76:6841-6844.
2. Baribaud, F., S. Pohlmann, G. Leslie, F. Mortari, and R. W. Doms. 2002. Quantitative expression and virus transmission analysis of DC-SIGN on moncyte-derived dendritic cells. J.Virol. 76:9135-9142.
3. Colmenares, M., A. Puig-Kroger, O. Muniz Pello, A. L. Corbi and L. Rivas. 2002. Dendritic-cell specific ICAM-3 grabbing nonintegrin (DC-SIGN, CD209), a C-type surface lectin in human dendritic cells, is a receptor for Leishmania amastigotes. J. Biol. Chem. 277:36766-36769.
4. Chan, L. and M. Guan. U.S. Pat. No. 5,824,506, October 1998.
5. Curtis, B. M., S. Scharnowske, and A. J. Watson. 1992. Sequence and expression of a membrane-associated C-type lectin that exhibits CD4-independent binding of human immunodeficiency virus envelope glycoprotein gp120. Proc. Natl. Acad. Sci. USA 89: 8356-8360.
6. Darden, J. M., V. R. Polonis, M. S. de Souza, S. Chantakulkij, A. E. Brown, D. L. Birx, K. Pattanapanyasat. 2000. A flow cytometric method for measuring neutralization of HIV-1 subtype B and E primary isolates. Cytokmetery; 40(2):141-150.
7. Eckels, K. H., W. E. Brandt, V. R. Harrison, J. M. McCown, and P. K. Russell. 1976. Isolation of a temperature-sensitive dengue-2 virus under conditions suitable for vaccine development. Infect. Immun. 14:1221-1227.
8. Flamand, M., F. Megret, S. Alcon, A. Talarmin, P. Despres, V. Deubel. U.S. Pat. No. 6,870,032, March 2005.
9. (I) Geijtenbeek, T. B., D. S. Kwon, R. Torensma, S. J. van Vliet, G. C. van Duijnhoven, J. Middel, I. L. Cornelissen, H. S. Nottet, V. N. KewalRamani, D. R. Littman, C. G. Fifdor, and Y. van Kooyk. 2000. DC-SIGN, a dendritic cell-specific HIV-1-binding protein that enhances trans-infection of T cells. Cell. 100:587-597.
10. (II) Geijtenbeek, T. B., R. Torensma, S. J. van Vliet, G. C. van Duijnhoven, G. J. Adema, Y. van Kooyk and C. G. Figdor. 2000. Identification of DC-SIGN, a novel dendritic cell-specific ICAM-3 receptor that support primary immune responses. Cell 100:575-585.
11. Geijtenbeek, T. B. H., S. J. van Vliet, E. A. Koppel, M. Sanchez-Hernandez, C. M. J. E. Vandenbroucke-Grauls, B. Appelmelk and Y. Van Kooyk. 2003. Mycobacteria target DC-SIGN to suppress dendritic cell function. J. Exp. Med. 197:7-17.
12. Gubler, D. J., S. Nalim, R. Tan, H. Saipan, and J. Sulianti Saroso. 1979. Vqariation in susceptibility to oral infection with dengue viruses among geographic strains of Aedes aegypti. Am. J. Trop. Med. Hyg. 28:1045-1052.
13. Gubler, D. I. 1988. Dengue. In The Arboviruses: Epidemiology and Ecology. T. P. Monath (ed.), CRC Press (Boca Raton), p223-260.
14. Halstead, S. B. 1997. Epidemiology of dengue and dengue hemorrhagic fever. In Dengue and Dengue Hemorrhagic Fever. D. J. Gubler and G. Kuno, editors. Cab international, London. 23-44.
15. Henchal, E. A. and J. R. Putnak. 1990. The dengue viruses. Clin. Microbiol. Rev. 3:376-396.
16. Hsu, E. C., F. Sarangi, C. Iorio, M. S. Sidhu, S. A. Udem, D. L. Dillehay, W. Xu, P. A. Rota, W. J. Bellini, and C. D. Richardson. 1998. A single amino acid change in the hemagglutinin protein of measles virus determines its ability t bind CD46 and reveals another receptor on marmoset B cells. J. Virol.; Vol 72:2905-2916.
17. Hsu, E. C., C. Iorio, F. Sarangi, A. A. Khine, C. D. Richardson. 2001. CDw150(SLAM) is a receptor for a lymphotropic strain of measles virus and may account for the immunosuppressive properties of this virus. Virology; 279(1):9-21.
18. Libraty, D. H., S. Pichyangkul, C. Ajariyakhajorn, T. P. Endy, and F. A. Ennis. 2001. Human dendritic cells are activated by dengue virus infection: enhancement by gamma interferon and implications for disease pathogenesis. J. Virol. 75::3501-3508.
19. Maeda, N., J. Nigou, J. L. Herrmann, M. Jackson, A. Amara, P. H. Lagrange, G. Puzo, B. Gicquel and O. Neyrolles. 2003. The cell surface receptor DC-SIGN discriminates between Mycobacterium species through selective recognition of the mannose caps on lipoarabinomannan. J. Biol. Chem. 278:5513-5516.
20. Marovich, M., G. Grouard-Vogel, M. Louder, M. Eller, W. Sun, S J. Wu, R. Putvatana, G. Murphy, B. Tassaneetrithep, T Burgess, et al. 2001. Human dendritic cells as targets of dengue virus infection. J. Investig. Dermatol. Symp. Proc. 6:219-224.
21. Mascola, R. R., M. K. Louder, C. Winter, R. Prabhakara, S. C. De Rosa, D. C. Douek, B. J. Hill, D. Gabuzda, and M. Roederer. 2002. Human Immunodeficiency virus type 1 neutralization measured by flow cytometeric quantitation of single-round infection of primary human T cells. J. Virol.; Vol 76:4810-4821.
22. Monath, T. P., and F. X. Heinz. 1996. Flaviviruses. In Fields Virology. B. N. Fields, D. M. Knipe and P. M. Howley, (eds.) Lippincott-Raven, Philadelphia. 961-1034.
23. Nimmannitya, S. 1996. Dengue and dengue haemorrhagic fever. In Manson's Tropical Diseases. G. C. Cook (eds.) W. B. Saunders Company, Ltd (London). 721-729.
24. Shu, P. Y., and J. H. Huang. 2004. Current advances in dengue diagnosis. Clin. Diagn. Lab. Immunol., 11(4):642-650.
25. Tailleux, L., 0. Schwartz, J.-L. Herrmann, E. Pivert, M. Jaackson, A. Amara, L. Legres, D. Dreher, L. P. Nicod, J. C. Gluckman, et al. 2003. DC-SIGN is the major mycobacterium tuberculosis receptor on human dendritic cells. J. Exp. Med. 197:121-127.
26. Thomas, S. J., D. Strickman, and D. W. Vaughn. 2003. Dengue epidemiology: virus epidemiology, ecology and emergence. Adv. Virus Res. 61:235-289.
27. World Health Organization. Dengue Hemorrhagic Fever: Diagnosis, Treatment and Control. Geneva: WHO, 1986.

Having described the invention, one of skill in the art will appreciate in the appended claims that many modifications and variations of the present invention are possible in light of the above teachings. It is therefore, to be understood that, within the scope of the appended claims, the invention may be practiced otherwise than as specifically described.

What is claimed is:

1. A dengue neutralization assay comprising determining the inhibition of binding of dengue virus to either or both dendritic cell-specific intracellular adhesion molecule dendritic cell specific 3-grabbing nonintegrin and liver/lymph node-specific intracellular adhesion molecule 3-grabbing nonintegrin by anti-dengue ligand.

2. The dengue neutralization assay of claim 1 further comprising the steps prior to said determining of inhibition of binding:
a. producing a series mixtures each of said mixture containing dengue virus and dilutions of either test ligand or positive anti-dengue or negative control ligand;
b. exposing said mixtures to either or both dendritic cell-specific intracellular adhesion molecule dendritic cell specific 3-grabbing nonintegrin and liver/lymph node-specific intracellular adhesion molecule 3-grabbing nonintegrin;
c. measuring binding of said dengue virus to either or both said dendritic cell-specific intracellular adhesion molecule dendritic cell specific 3-grabbing nonintegrin and liver/lymph node-specific intracellular adhesion molecule 3-grabbing nonintegrin from said mixtures containing said dendritic cell-specific intracellular adhesion molecule dendritic cell specific 3-grabbing nonintegrin and liver/lymph node-specific intracellular adhesion molecule 3-grabbing nonintegrin.
d. using said binding measurements in order to determine percent neutralization of said test ligand.

3. The method of claim 1, wherein said anti-dengue ligand is patient serum antibody.

4. The method of claim 1, wherein said anti-dengue ligand is a potential anti-dengue pharmaceutical.

5. The method of claim 1, wherein either or both said dendritic cell-specific intracellular adhesion molecule dendritic cell specific 3-grabbing nonintegrin and liver/lymph node-specific intracellular adhesion molecule 3-grabbing nonintegrin is expressed on a cell line transfected with DNA encoding either or both of dendritic cell-specific intracellular adhesion molecule dendritic cell specific 3-grabbing nonintegrin and liver/lymph node-specific intracellular adhesion molecule 3-grabbing nonintegrin.

6. The method of claim 1, wherein said dendritic cell-specific intracellular adhesion molecule dendritic cell specific 3-grabbing nonintegrin and liver/lymph node-specific intracellular adhesion molecule 3-grabbing nonintegrin are attached to a solid surface.

7. The method of claim 2, wherein said measuring step is by flow cytometry.

8. The method of claim 2, wherein said measuring step includes the following steps:
a. applying said sera exposed transfected cells to microscope slides;
b. permeabilizing transfected cells;
c. exposing said permeabilized cells to anti-dengue virus probe antibody conjugated to fluorochrome or exposing said permeabilized cells to anti-dengue virus antibody and subsequently exposing said permeabilized cells to fluorochrome-conjugated secondary antibody;
d. detecting the binding of said probe antibody by microscopy.

9. The method of claim 2, wherein said measuring step is by enzyme-linked immunosorbent assay.

10. The method of claim 5, wherein said tumor cell line is Raji B cells.

11. The method of claim 7, wherein said solid surface is a microbead.
